(19) United States
(12) Patent Application Publication
(10) Pub. No.: US 2011/0179516 A1
(43) Pub. Date: Jul. 21, 2011

(54) PLANTS PRODUCING 2N POLLEN

(76) Inventors: Raphael Mercier, Fonteray Le Fleuny (FR); Isabelle D'Erfurth, Quetigny (FR); Laurence Cromer, Clamart (FR)

(21) Appl. No.: 13/002,982

(22) PCT Filed: Jul. 7, 2009
(86) PCT No.: PCT/IB2009/006590

§ 371 Date: Apr. 4, 2011

(30) Foreign Application Priority Data
Jul. 8, 2008 (EP) 08290672.8

(51) Int. Cl. A01H 1/08; C12N 15/82; A01H 5/00

(52) U.S. Cl. 800/276; 800/285; 435/320.1; 800/298; 800/312; 800/320.2; 800/320; 800/317.4; 800/317.2; 800/322; 800/315; 800/320.3

(57) ABSTRACT

The invention relates to methods for obtaining plants that produce 2n pollen. These plants are useful in plant breeding.

The invention relates to plants that produce 2n pollen, and to their use in plant breeding.

Polyploidy is the condition of organisms that have more than two sets of chromosomes. It has played a pervasive role in the evolution, adaptation and speciation of many eukaryotes, including yeasts, insects, amphibians, reptiles, fishes and vertebrates (OTTO, Cell 131, 452-62, 2007).

Polyploidy is particularly prominent in plants; it is estimated that 95% of ferns are polyploids and that almost all angiosperms have experienced at least one round of whole genome duplication during the course of their evolution (CUI, et al. Genome Res 16, 738-49, 2006). In addition, many important crop plants, such as potato, cotton, oilseed rape, alfalfa and wheat are current polyploids, while others, such as maize, soybean, and cabbage, retain the vestiges of ancient polyploid events (GAUT & DOEBLEY, Proc Natl Acad Sci USA, 94, 6809-14, 1997; LYSAK et al., Genome Res 15, 516-25, 2005; SCHLUETER, et al. BMC Genomics, 8, 330, 2007). Even plants with small genomes, such as Arabidopsis thaliana, have been impacted by polyploidy (BLANC et al., Plant Cell 12, 1093-101, 2000).

The mechanisms responsible for the formation of polyploids in plants are still poorly understood. However, it is now believed that 2n gametes are the major route for polyploidy formation and that they drive gene flow to occur from the diploid progenitors to the new polyploid species. (BRETAGNOLLE & THOMPSON, New Phytologist 129, 1-22, 1995; RAMSEY & SCHEMSKE, Annual Reviews of Ecology and Systematics 29, 467-501, 1998).

2n gametes (also known as unreduced gametes or diplogametes) are gametes having the somatic chromosome number rather than the gametophytic chromosome number. They have been shown to be useful for the genetic improvement of several crops (for review, cf. for instance RAMANNA & JACOBSEN, Euphytica 133, 3-18, 2003).

Given their tremendous importance in evolution and agronomy, 2n gametes have focused considerable attention. (VEILLEUX, Plant Breeding Reviews 3, 252-288, 1985) and BRETAGNOLLE & THOMPSON (1995, cited above) provided exhaustive review of the meiotic aberrations that can generate diplogametes. The best documented and described cytological abnormalities leading to 2n gametes formation include abnormal cytokinesis, the skip of the first or second meiotic division, or abnormal spindle geometry. Co-orientation of 2nd division spindles (parallel spindles or fused spindles) is perhaps the most common of the mechanisms responsible for the formation of 2n spores, and is the main mechanism of 2n pollen formation in potato (CARPUTO et al. Genetics 163, 287-94, 2003). Moreover the mode of formation of 2n gametes has a direct impact on their genetic composition. For example, parallel or fused spindles lead to gametes that are completely heterozygous up to the first crossover on each pair of homologues and that are therefore genetically equivalent to those resulting from the absence of first division (apart from the segregation of recombinant chromatids beyond the first crossover). These gametes are different from those resulting from premature or abnormal cytokinesis that usually contain the two sisters chromatids of every chromosome, and that are therefore genetically equivalent to the gametes resulting from the absence of second meiotic division.

Although environmental factors can affect the frequency of 2n gametes, the ability to produce 2n gametes is heritable and has therefore a strong genetic basis (RAMSEY & SCHEMSKE, 1998, cited above). The genetic determination of 2n pollen production has been studied in detail in several species and usually fit the segregation of a major locus in a background of polygenic variation. In most instances, the capacity to form 2n gametes was found to be controlled by a monogenic recessive allele, while the expression of this phenotype was modulated by several other loci (reviewed in BRETAGNOLLE & THOMPSON, 1995, cited above) and external environment.

So far, none of the genes that contribute to 2n gametes production has been identified and characterized at the molecular level, and this lack of information has hampered a broader use of these gametes in man-assisted breeding programs.

The inventors have now characterized in the model plant Arabidopsis thaliana, a gene implicated in the formation of 2n gametes in plants. This gene will be hereinafter designated AtPS1 (for Arabidopsis thaliana parallel spindles). The inventors have found that inactivation of AtPS1 generates diploid male spores, giving rise to viable diploid pollen grains and to spontaneous triploid plants in the progeny. The sequence of the AtPS1 gene of Arabidopsis thaliana, is available in the TAIR database under the accession number AT1g34355. This gene encodes a protein of 1477 an, whose sequence is represented in the enclosed sequence listing as SEQ ID NO: 1. The AtPS1 gene is conserved in higher plants. A search in the sequence databases allowed to identify orthologs of the AtPS1, for instance in Populus trichocarpa (SEQ ID NO:2), Oryza sativa (SEQ ID NO:3), Vitis vinifera (SEQ ID NO:4), Glycine max, (SEQ ID NO:5 and 6) Sorghum bicolour (SEQ ID NO:7), Zea mays (SEQ ID NO:8), (Solanum lycopersicum (SEQ ID NO:9), Medicago truncatula (SEQ ID NO:10), Solanum tuberosum (partial sequence represented as SEQ ID NO:11), Helianthus argophyllus (partial sequence represented as SEQ ID NO:12), Malus×domestica (partial sequence represented as SEQ ID NO:13), and Triticum aestivum (partial sequence represented as SEQ ID NO:14). These proteins bear two domains, a PINc domain (InterPro: IPR006596) which is predicted to play a role in nucleotide-binding, potentially being found in RNases, and a FHA (ForkHead Associated) domain (InterPro: IPR000253) a phosphopeptide recognition domain found in many regulatory proteins.

The invention thus provides a method for obtaining a plant producing 2n pollen, wherein said method comprises the inhibition in said plant of a protein hereinafter designated as PS1 protein, wherein said protein:
comprises within its N-terminal region (preferably within its 300 N-terminal amino-acids, more preferably within its 250 N-terminal aminoacids, and still more preferably within its 200 N-terminal aminoacids), a domain having at least 40%, and by order of increasing preference, at least 45, 50, 55, 60, 65, 70, 75, 80, 85, 90, 95 or 98% sequence identity, or at least 60%, and by order of increasing preference, at least, 65, 70, 75, 80, 85, 90, 95 or 98% sequence similarity with the FHA domain of the AtPS1 protein (amino acids 64-132 of SEQ ID NO: 1);
comprises within its C-terminal region (preferably within its 350 N-terminal amino-acids, more preferably within its 300 N-terminal aminoacids, and still more preferably within its 250 N-terminal aminoacids), a domain having at least 40%, and by order of increasing preference, at least 45, 50, 55, 60, 65, 70, 75, 80, 85, 90, 95 or 98% sequence identity, or at least 60%, and by order of increasing preference, at least, 65, 70, 75, 80, 85, 90, 95 or 98% sequence similarity with the PINc domain of the AtPS1 protein (amino acids 1237-1389 of SEQ ID NO: 1).

Unless otherwise specified, the protein sequence identity and similarity values provided herein are calculated using the BLASTP program under default parameters. Similarity calculations are performed using the scoring matrix BLOSUM62.

By way of non-limitative examples of representative PS1 proteins of various plant families, one can cite, besides the AtPS1 of SEQ ID NO: 1, the Populus trichocarpa PS1 protein of SEQ ID NO: 2, the Oryza sativa PS1 protein of SEQ ID NO: 3, the Vitis vinifera PS1 protein of SEQ ID NO:4, the Glycine max PS1 proteins of SEQ ID NO:5 and 6, the Sorghum bicolour PS1 protein of SEQ ID NO:7, the Zea mays PS1 protein of SEQ ID NO:8, the Solanum lycopersicum PS1 protein of SEQ ID NO:9, the Medicago truncatula PS1 protein of SEQ ID NO:10, the Solanum tuberosum PS1 protein (partial sequence SEQ ID NO:11), the Helianthus argophyllus PS1 protein (partial sequence SEQ ID NO:12), the Malus×domestica PS1 protein (partial sequence SEQ ID NO:13), and the Triticum aestivum PS1 protein (partial sequence as SEQ ID NO:14).

The inhibition of a PS1 protein can be obtained either by abolishing, blocking, or decreasing its function, or advantageously, by preventing or down-regulating the expression of its gene.

By way of example, inhibition of said PS1 protein can be obtained by mutagenesis of the corresponding gene or of its promoter, and selection of the mutants having partially or totally lost the PS1 protein activity. For instance, a mutation within the coding sequence can induce, depending on the nature of the mutation, the expression of an inactive protein, or of a protein with impaired activity; in the same way, a mutation within the promoter sequence can induce a lack of expression of said PS 1 protein, or decrease thereof.

Mutagenesis can be performed for instance by targeted deletion of the PS1 coding sequence or promoter, or of a portion thereof, or by targeted insertion of an exogenous sequence within said coding sequence or said promoter. It can also be performed by inducing random mutations, for instance through EMS mutagenesis or random insertional mutagenesis, followed by screening of the mutants within the PS1 gene. Methods for high throughput mutagenesis and screening are available in the art. By way of example, one can mention TILLING (Targeting Induced Local Lesions IN Genomes, described by McCallum et al., 2000). Among the progeny of the mutants having a mutation within the PS1 gene, the plants which are homozygous for the mutation have the ability to produce 2n pollen; these plants can be identified among on the basis of their phenotypic characteristics, for instance the formation of at least 5%, preferably at least 10%, and more preferably at least 20% of dyads as a product of male meiosis.

Advantageously, the inhibition of said PS1 protein is obtained by silencing of the corresponding PS1 gene. Methods for gene silencing in plants are known in themselves in the art. For instance, one can mention by antisense inhibition or co-suppression, as described by way of example in U.S. Pat. Nos. 5,190,065 and 5,283,323. It is also possible to use ribozymes targeting the mRNA of said PS1 protein.

Preferred methods are those wherein gene silencing is induced by means of RNA interference (RNAi), using a silencing RNA targeting the PS1 gene to be silenced. Various methods and DNA constructs for delivery of silencing RNAs are available in the art.

A “silencing RNA” is herein defined as a small RNA that can silence a target gene in a sequence-specific manner by base pairing to complementary mRNA molecules. Silencing RNAs include in particular small interfering RNAs (siRNAs) and microRNAs (miRNAs).

Initially, DNA constructs for delivering a silencing RNA in a plant included a fragment of 300 bp or more (generally 300-800 bp, although shorter sequences may sometime induce efficient silencing) of the cDNA of the target gene, under transcriptional control of a promoter active in said plant. Currently, the more widely used silencing RNA constructs are those that can produce hairpin RNA (hpRNA) transcripts. In these constructs, the fragment of the target gene is inversely repeated, with generally a spacer region between the repeats (for review, cf. WATSON et al., 2005). One can also use artificial microRNAs (amiRNAs) directed against the PS1 gene to be silenced (for review about the design and applications of silencing RNAs, including in particular amiRNAs, in plants cf. for instance OSSOWSKI et al., (Plant J., 53, 674-90, 2008).

The present invention provides chimeric DNA constructs for silencing a PS1 gene, including in particular expression cassettes for hpRNA, or amiRNA targeting the PS1 gene.

An expression cassette of the invention may comprise for instance:
a promoter functional in a plant cell;
a DNA construct of 200 to 1000 bp, preferably of 300 to 900 bp, comprising a fragment of a cDNA encoding a PS1 protein or of its complementary, or having at least 95% identity, and by order of increasing preference, at least 96%, 97%, 98%, or 99% identity with said fragment, said DNA sequence being placed under transcriptional control of said promoter.

According to a preferred embodiment of the invention, an expression cassette for a hpRNA
a promoter functional in a plant cell,
a DNA construct which is capable, when transcribed, of forming a hairpin RNA targeting the PS1 gene;

said DNA construct being placed under transcriptional control of said promoter.

Generally, said hairpin DNA construct comprises: i) a first DNA sequence of 200 to 1000 bp, preferably of 300 to 900 bp, consisting of a fragment of a cDNA encoding a PS1 protein, or having at least 95% identity, and by order of increasing preference, at least 96%, 97%, 98%, or 99% identity with said fragment; ii) a second DNA sequence that is the complementary of said first DNA, said first and second sequences being in opposite orientations and ii) a spacer sequence separating said first and second sequence, such that these first and second DNA sequences are capable, when transcribed, of forming a single double-stranded RNA molecule. The spacer can be a random fragment of DNA. However, preferably, one will use an intron which is spliceable by the target plant cell. Its size is generally 400 to 2000 nucleotides in length.

According to another preferred embodiment of the invention, an expression cassette for a amiRNA targeting the PS1 gene comprises:
a promoter functional in a plant cell,
a DNA construct which is capable, when transcribed, of forming an amiRNA targeting the PS1 gene;

said construct being placed under transcriptional control of said promoter.

A large choice of promoters suitable for expression of heterologous genes in plants is available in the art.

They can be obtained for instance from plants, plant viruses, or bacteria such as Agrobacterium. They include constitutive promoters, i.e. promoters which are active in most tissues and cells and under most environmental conditions, as well as tissue-specific or cell-specific promoters which are active only or mainly in certain tissues or certain cell types, and inducible promoters that are activated by physical or chemical stimuli, such as those resulting from nematode infection.

Non-limitative examples of constitutive promoters that are commonly used in plant cells are the cauliflower mosaic virus (CaMV) 35S promoter, the Nos promoter, the rubisco promoter, the Cassava vein Mosaic Virus (CsVMV) promoter.

Organ or tissue specific promoters that can be used in the present invention include in particular promoters able to confer meiosis-associated expression, such as the DMC1 promoter (KLIMYUK & JONES, Plant J, 11, 1-14, 1997); one can also use the endogenous promoter of PS1.

The DNA constructs of the invention generally also include a transcriptional terminator (for instance the 35S transcriptional terminator, or the nopaline synthase (Nos) transcriptional terminator).

The invention also includes recombinant vectors containing a chimeric DNA construct of the invention. Classically, said recombinant vectors also include one or more marker genes, which allow for selection of transformed hosts.

The selection of suitable vectors and the methods for inserting DNA constructs therein are well known to persons of ordinary skill in the art. The choice of the vector depends on the intended host and on the intended method of transformation of said host. A variety of methods for genetic transformation of plant cells or plants are available in the art for many plant species, dicotyledons or monocotyledons. By way of non-limitative examples, one can mention virus mediated transformation, transformation by microinjection, by electroporation, microprojectile mediated transformation, Agrobacterium mediated transformation, and the like.

The invention also provides a host cell comprising a recombinant DNA construct of the invention. Said host cell can be a prokaryotic cell, for instance an Agrobacterium cell, or a eukaryotic cell, for instance a plant cell genetically transformed by a DNA construct of the invention. The construct may be transiently expressed; it can also be incorporated in a stable extrachromosomal replicon, or integrated in the chromosome.

The invention also provides a method for producing a transgenic plant able to produce 2n pollen, said method comprising the steps consisting of:
transforming at least one plant cell with a vector containing a DNA construct of the invention;
cultivating said transformed plant cell in order to regenerate a plant having in its genome a transgene containing said DNA construct.

The invention also provides plants genetically transformed by a DNA construct of the invention. Preferably, said plants are transgenic plants, wherein said construct is contained in a transgene integrated in the plant genome, so that it is passed onto successive plant generations. The expression of said chimeric DNA constructs, resulting in a down regulation of the PS1 protein, provides to said transgenic plant the ability to produce 2n pollen.

The invention also encompasses a method for producing 2n pollen, wherein said method comprises cultivating a plant obtained by a method of the invention and recovering the pollen produced by said plant. Preferably said pollen comprises at least 10%, more preferably at least 20%, and by order of increasing preference, at least 30%, 40%, 50%, or 60% of viable 2n pollen grains.

The 2n pollen produced by the plants of the invention is useful in particular in plant breeding, for producing polyploids plants (for instance sterile triploids), or to allow crosses between plants of different ploidy level.

The present invention applies to a broad range of monocot- or dicotyledon plants of agronomical interest. By way of non-limitative examples, one can mention potato, tomato, alfalfa, sugar cane, sweet potato, manioc, blueberry, clover, soybean, ray-grass, banana, melon, watermelon or ornamental plants such as roses, lilies, tulips, narcissus.

Foregoing and other objects and advantages of the invention will become more apparent from the following detailed description and accompanying drawings. It is to be understood however that this foregoing detailed description is exemplary only and is not restrictive of the invention.

### DESCRIPTION OF THE DRAWINGS

FIG. 1: AtPS1 Gene Structure

Intron/exon structure of the AtPS1 gene and location of the three different T-DNA insertions (triangles). The primers used are indicated below the gene diagram.

FIG. 2: Multiple Sequence Alignment Representing Segments of Highest Sequence Conservation Among Plant AtPS1 Proteins

Full-length AtPS1 proteins of Arabidopsis thaliana (At: NP_{—}564445) Populus trichocarpa (Pt: jgi_{—}592219), Oryza sativa (Os: NP_{—}001065865), Vitis vinifera (Vv: CAN81434_mod), Glycine max (Gm: jgi_scaffold_{—}143 and jgi_scaffold_{—}131), Sorghum bicolour (Sb: jgi_{—}5039668), and Zea mays (Zm: EST_{—}10287.m000022) were aligned and segments of highest conservation were identified using plotcon (EMBOSS package).

The sequences shown in this alignment are derived from those represented in the enclosed sequence listing under SEQ ID NO: 1 to 8 by removal of the non-conserved sequence segments from the alignment. The length of these deleted regions is indicated in box brackets in the corresponding position of the alignment. Domain hits based on a comparison against the Interpro domain database (AMID: 18428686) are indicated by gray boxes above the alignment, and the hit-indicators are extended to include adjacent segments of sequence and structure conservation. In addition, a short motif identified as a repeat element C in Arabidopsis thaliana AtPS1, is marked using white boxes.

FIG. 3: Wild Type and Atps1 Mutants Meiotic Products Analysis.

A: Photos of meiotic products in wild type and Atps1 mutants. Scale bar=10 μm. B: Quantification of meiotic products in Ws-4 (n=92), Col-0 (n=212), Atps1-3 (n=436), Atps1-1 (n=1125), Atps1-2 (n=554), the Atps1-1/Atps1-3 F1 (n=283) and Atps1-1/Atps1-2 F1 (n=252).

FIG. 4: Meiotic Chromosome Spreads of Wild Type and Atps1.

A-F: Wild type meiotic chromosome spreads. A: pachytene. B: metaphase I. C: anaphase I. D: metaphase II. E: Anaphase II F: telophase II. G-O Atps1-1 meiosis. G: pachytene. H: metaphase I I: anaphase I J-L: metaphase H. M-O: Anaphase II. M: dyad. N: triad. O: tetrad. Scale bar=10 μm.

FIG. 5: Immuno-Staining of Meiosis II Spindles in Wild Type and Atps1-1 Mutant.

A, B, C: Wild type spindles at metaphase II, anaphase II and telophase II, respectively. D to L: Atps1-1 meiocyte at metaphase II/anaphase II. Chromosomes were stained by propidium iodide or DAPI and microtubules by immunolocalisation. Scale bar=5 μm.

FIG. 6: Meiotic Chromosome Spreads of Atspo11-1 and Atps1-1/Atspo11-1 Double Mutant.

A to F: Atspo11-1 meiotic chromosome spreads. A: metaphase I. B and C: anaphase I. D: metaphase II. E: Unbalanced Tetrad. F: Polyad. G to R: Atps1-1/Atspo11-1 meiosis. G: metaphase I. H and I: anaphase I. J: metaphase II. K: Anaphase II. L and M: Balanced dyad II. N and O: Triad. P and Q: Unbalanced tetrad. R: Polyad. Scale bar=10 μm.

FIG. 7: Genotype of Offspring of Atps1 Mutants.

Diploid and triploid offspring of the Atps1-1(Col-0)/Atps1-3(Ws-4) ♀×Ler ♀ cross was genotyped for several genetic markers. For each marker plants bearing only the Col-0 allele are in medium grey, plants bearing only the Ws-4 allele are in light grey and plants bearing both the Col-0 and Ws-4 alleles are in dark grey. The Ler alleles are present in all the plants because it was used as the female parent in the cross. The position of each marker and the centromeres are indicated along the chromosomes.

FIG. 8. Meiotic Products of Wild Type, atps1-1 Mutant and RNAI35AtPS1#6 Plant.

FIG. 9. Expression of AtPS1 in Three RNAI35AtPS1 Plants.

### EXAMPLES

### Experimental Procedures

### Plant Material and Growth Conditions.

The wild-type references plant material used in this study were A. thaliana accession Columbia (Col-0) and Wassilewskija (Ws-4). The T-DNA insertion mutant lines N578818 (ALONSO et al., Science, 301, 653-7, 2003) (Atps1-1), N851945 (WOODY et al., J Plant Res 120, 157-65, 2007) (Atps1-2), N646172 (Atspo11-1-3: STACEY et al. Plant Journal 48, 206-216 2006) obtained from the European Arabidopsis stock center (SCHOLL et al., Plant Physiol. 124, 1477-80, 2000) and EQM96 (Atps1-3) from the Versailles T-DNA collection (SAMSON et al., Nucleic Acids Res 30, 94-7, 2002) were used.

Arabidopsis plants were cultivated in a greenhouse or growth chamber under the following conditions: photoperiod 16 h day/8 h night; temperature 20° C. day and night; humidity 70%. For germination assay and cytometry experiments Arabidopsis were cultivated in vitro on Arabidopsis medium (ESTELLE et al., Mol. Gen. Genet. 206, 200-206, 1987) at 21° C. with a photoperiod of 16h day/8h night, and 70% of hygrometry.

### Genetic Analysis.

Genotyping of T-DNA insertion mutants was done by PCR (30 cycles of 30s at 94° C., 30s at 56° C. and 1 min at 72° C.) using two couple of primers. For each line the first couple designated is specific of the wild type locus and second couple is specific of the T-DNA insertion.

Atps1-3: EQM96L (5′ACATCTCCCTTGTCGTAAC3′: SEQ ID NO:15) and EQM96U (5′ATCTCTCAATCGTTCGTTC3′: SEQ ID NO:16); EQM96L and tag3 (5′CTGATACCAGACGTTGCCCGCATAA3′: SEQ ID NO:17).

### Atps1-1: N578818U2 (5′TCGGAGTCACGAAGACTATG3′: SEQ ID NO:18) and N578818L (5′CAGTCTCACTGATTATTCCTG 3′: SEQ ID NO:19); N578818U2 and LbSalk2 (5′GCTTTCTTCCCTTCCTTTCTC 3′: SEQ ID NO:20).

### Atps1-2: N851945U (5′AAGGCTGATATTCTGATTCAT3′: SEQ ID NO:21) and N851945L (5′ CTCTTGTTGGTCCGTATCTTA3′: SEQ ID NO:22); N851945U and P745 (5′AACGTCCGCAATGTGTTATTAAGTTGTC3′: SEQ ID NO:23).

spo11-3: N646172U (5′ AATCGGTGAGTCAGGTTTCAG3′: SEQ ID NO:24) and N646172L (5′ CCATGGATGAAAGCGATTTAG3′: SEQ ID NO:25); N646172L/LbSalk2.

Double spo11/Atps1 mutants were obtained as described in VIGNARD et al. (PLoS Genet, 3, 1894-906, 2007)

### Genetic Markers Used to Genotype Atps1-1/Atps1-3×Ler F1 Triploid and Diploid Plants

Microsatellite msat1.29450 (located on chromosome I at position 29450001) was amplified (Tm=57° C.) using 5′TCCTTTCATCTTAATATGC3′ (SEQ ID NO:26) and 5′TCTGTCCACGAATTATTTA3′ (SEQ ID NO:27) primers. Microsatellite Msat4.35 (Tm=58° C.) (located on chromosome 4 at position 7549125) was amplified using 5′ CCCATGTCTCCGATGA3′ (SEQ ID NO:28) and 5′ GGCGTTTAATTTGCATTCT3′ (SEQ ID NO:29) primers. Microsatellite NGA151 (Tm=58° C.) (located on chromosome 5 at position 4669932) was amplified using 5′GTTTTGGGAAGTTTTGCTGG3′(SEQ ID NO:30) and 5′ CAGTCTAAAAGCGAGAGTATGATG3′ (SEQ ID NO:31) primers. The 2 primer pairs specific for the Atps1-1 and Atps1-3 TDNA borders were used as a centromeric marker of the chromosome 1. CAPS markers Seqf16k23 (physical position: 14481813) and CAPSK4 51 (physical position: 5078201) were used as centromeric markers for chromosome 1 and 4, respectively.

CAPS Seqf16k23 is amplified (Tm=60° C.) using 5′GAGGATACCTCTTGCTGATTC3′ (SEQ ID NO:32) and 5′CCTGGCCTTAGGAACTTACTC3′ (SEQ ID NO:33) primers and observed after TaqI digestion.

CAPS CAPSK4 51 is amplified (Tm=60° C.) using 5′CAATTTGTTACCAGTMGCAG3′ (SEQ ID NO:34) and 5′TGAGTTTGGTTTTTTGTTATTAGC3′ (SEQ NO:35) primers and observed after MnlI digestion.

PCR conditions: 40 cycles of 20s at 94° C., 20s at Tm and 30s at 72° C.

### RT-PCR

Arabidopsis total RNA were extracted using the QIAGEN RNA kit.

Reverse transcription were done on 5 μg of total RNA using an oligo(dT) (ALTSCHUL et al., Nucleic Acids Res 25, 3389-402, 1997) as primer. The RevertAid™ M. MuLV Reverse Transcriptase enzyme (FERMENTAS) was used according to the instructions of the manufacturer. RT-PCR were done on 1 μl of cDNA using the pAtpsF 5′GCCTTTTCAACCTCTACTTG3′ (SEQ ID NO:36) and pAtpsR 5′ATGGTGATAGATGATGATGATAC3′ (SEQ ID NO:37) primers under the following conditions: 30 cycles of 30 s at 94° C., 30 s at 56° C. and 1 min at 72° C.

### Cytology and Flow Cytometry:

Final meiotic products were observed as described in AZUMI et al., (Embo J, 21, 3081-95., 2002) and viewed with a conventional light microscope with a 40× dry objective. Chromosomes spreads and observations were carried out using the technique described in MERCIER et al., (Biochimie, 83, 1023-28, 2001). The DNA fluorescence of spermatic pollen nuclei was quantified using open LAB 4.0.4 software. For each nucleus the surrounding background was calculated and subtracted from the global fluorescence of the nucleus. Meiotic spindles were observed according to the protocol described in MERCIER et al., (Genes Dev, 15, 1859-71, 2001) except that the DNA was counter-stained with DAPI.

Observations were made using an SP2 leica confocal microscope. Images were acquired with a 63× water objective in xyz and 3D reconstructions were made using leica software. Projections are shown. Cells were imaged at excitation 488 nm and 405 nm with AlexaFluor488 and DAPI respectively. Arabidopsis genome sizes were measured as described in MARIE & BROWN, (Biol Cell, 78, 41-51, 1993) using tomato Lycopersicon esculentum cv “Montfavet” as the standard. (2C=1.99 pg, % GC=40.0%)

### Example 1

### Identification of the Arabidopsis AtPS1 Gene and atps1 Mutations

As a part of a screen for meiotic mutants, the At1g34355 gene was selected as a potential meiotic gene according to its expression profile. Using the Expression Angler tool (TOUFIGHI et al. Plant J 43, 153-63, 2005) with the AtGenExpress tissue set (SCHMID et al., Nat Genet 37, 501-6, 2005) this gene was found to be co-regulated with genes known to be involved in meiosis (AtMER3, AtDMC1, SDS, AtMND1, AtHOP2), with the highest expression level in shoot apex and young flower buds.

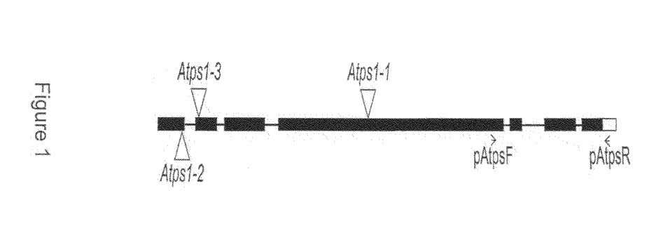

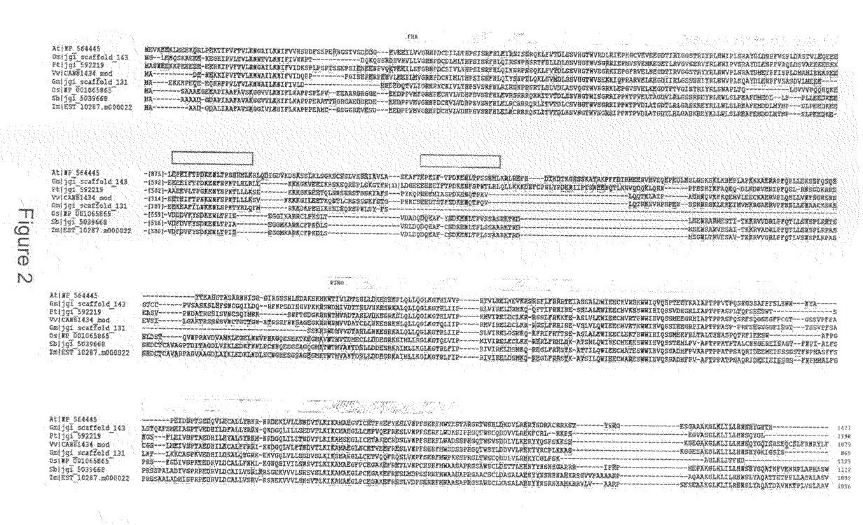

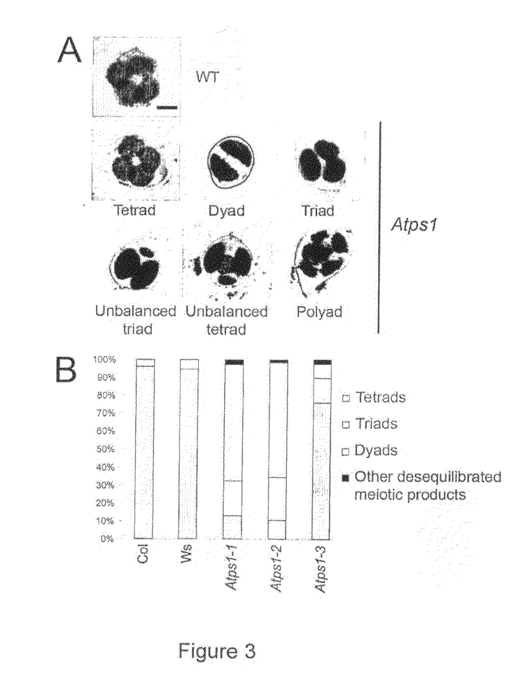

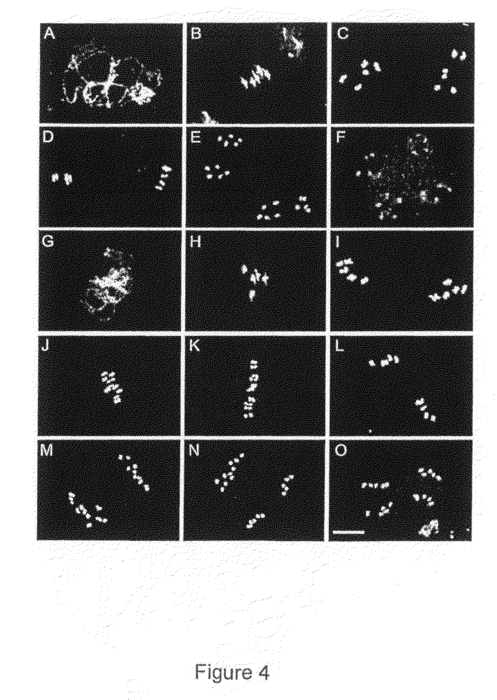

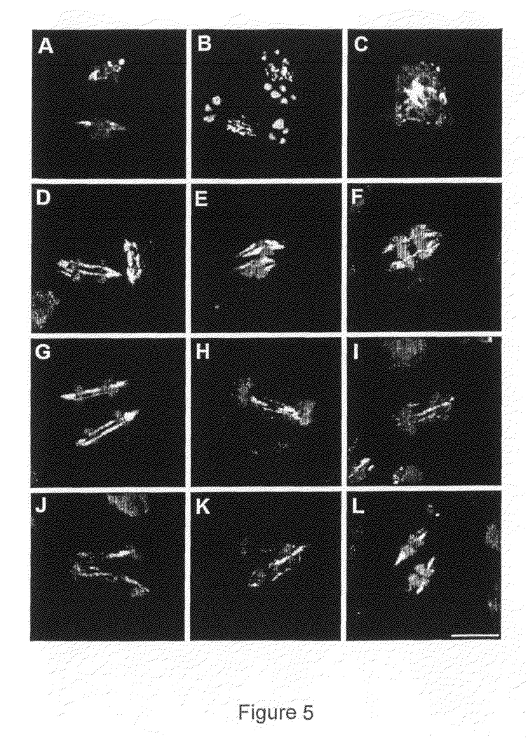

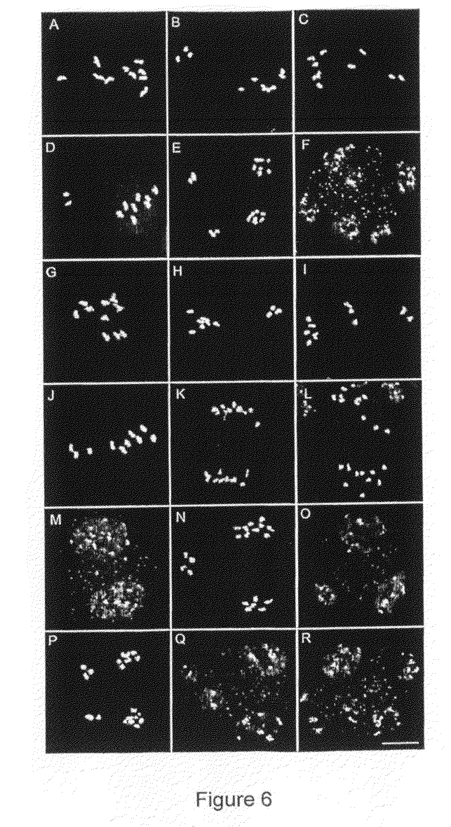

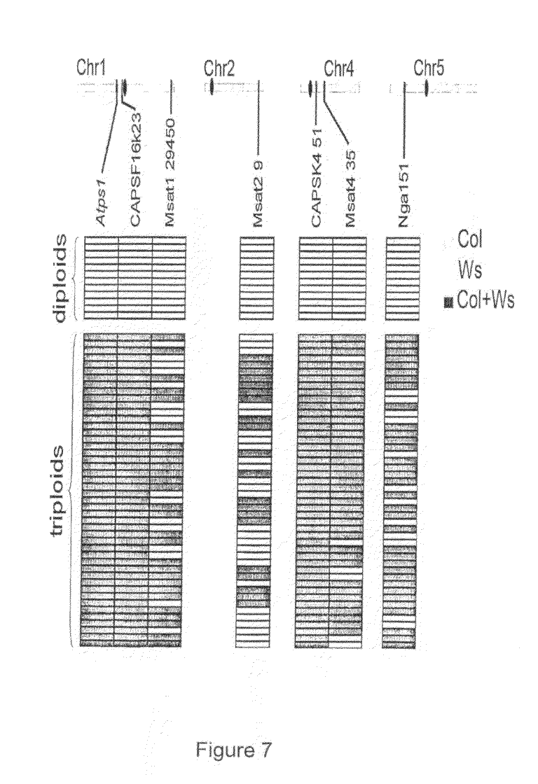

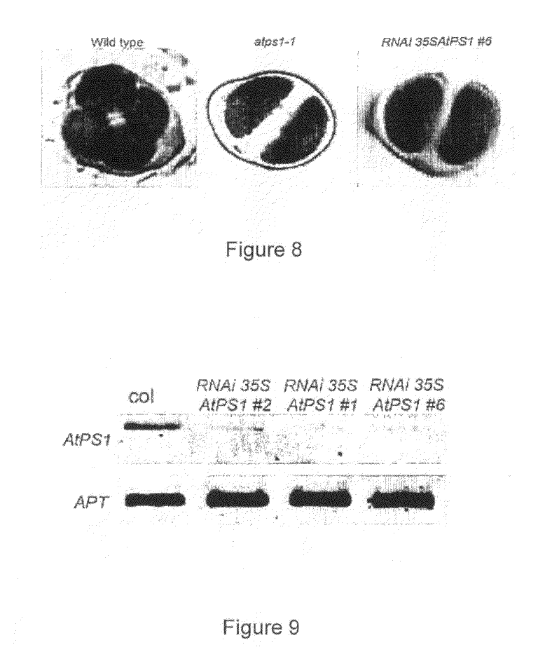

We amplified the AtPS1 cDNA (EU839993) by RT-PCR on bud cDNA and sequencing confirmed that it is identical to that predicted in the databases (NM_{—}103158). The AtPS1 gene contains 7 exons and 6 introns (FIG. 1) and encodes a protein of 1477 amino acids. BlastP and Psi-Blast (ALTSCHUL et al., Nucleic Acids Res, 25, 3389-402, 1997) analyses showed that the AtPS1 protein is conserved throughout the plant kingdom and contains two highly conserved regions (FIG. 2).

An FHA domain (forkhead associated domain) was predicted at the N terminus by CD searches (MARCHLER-BAUER & BRYANT, Nucleic Acids Res, 32, W327-31, 2004), while the C terminal conserved region shows similarity to a PIN Domain. These domains are separed by a compositionally biased sequence of variable length.

An FHA domain is a phosphopeptide recognition motif implicated in protein-protein interactions and is found in a diverse range of proteins involved in numerous processes including intracellular signal transduction, cell cycle control, transcription, DNA repair and protein degradation (DUROCHER & JACKSON, FEBS Lett, 513, 58-66, 2002). The PIN domain was predicted to have RNA-binding properties often associated with Rnase activity (CLISSOLD & PONTING, Curr Biol, 10, R888-90, 2000), and this has now been experimentally confirmed (GLAVAN et al., Embo J, 25, 5117-25, 2006). Accordingly, several PIN domain containing proteins are involved in Rnai, RNA maturation, or RNA decay. We could not identify non-plant proteins with significant similarity to AtPS1 (apart from the FHA and the PIN domains) or which contained both a FHA and a PIN domain.

While a single AtPS1 representative is usually found per species, gene duplication events have occurred in individual lineages such as in Glycine max.

We investigated the role of the AtPS1 gene by isolating and characterising a series of allelic mutants, identified in several public T-DNA insertion line collections (ALONSO et al., Science, 301, 653-7, 2003; SAMSON et al., Nucleic Acids Res, 30, 94-7, 2002; WOODY et al., J Plant Res, 120, 157-65, 2007). The Atps1-1 (SALK_{—}078818) and Atps1-2 (WiscDsLox342F09) lines were obtained from the European Arabidopsis stock centre (SCHOLL et al., Plant Physiol, 124, 1477-80, 2000) and are in a Columbia (Col-0) background. The insertions are in the fourth exon and first intron, respectively (FIG. 1). The Atps1-3 (FLAG_{—}456A09) insertion is from the Versailles T-DNA collection (SAMSON et al., Nucleic Acids Res, 30, 94-7, 2002) and is in a Wassilewskija (Ws-4) background, it is located in the second exon (FIG. 1). RT-PCR was carried out using the pAtpsF/pAtpsR primers (FIG. 1) on RNA from the Atps1-3 and Atps1-1 mutants and no detectable levels of the AtPS1 transcript were amplified, indicating that these two alleles are null. When the same primers were used on RNA from the Atps1-2 mutant normal expression levels of this region of the AtPS1 transcript were observed (data not shown). Nevertheless, the phenotype analysis described below strongly suggests that this third allele is also null.

### Example 2

### Meiosis in Atps1 Mutants Generates Dyads at High Frequency

In A. thaliana, the product of male meiosis is a group of four spores, organized in a tetrahedron, called tetrad. Our observation of the male meiotic products in wild type revealed almost exclusively tetrads (FIG. 3). In rare occasions, (13/304) groups of three spores were also observed and certainly resulted from occasional superposition of spores. In contrast, the observation of meiotic products of the three independent Atps1 mutants revealed the presence of a high frequency of dyads, triads and other uneven meiotic products (FIGS. 3A and 3B). The Atps1-1 and Atps1-2 mutants produced a majority of dyads (˜65%). The Atps1-3 mutant phenotype appeared to be weaker and only 8% of its meiotic products were dyads. Complementation tests realized between Atps1-1 and Atps1-2 and between Atps1-3 and Atps1-1 showed that these mutations are allelic (FIG. 3B), and thus demonstrated that the disruption of the AtPS1 gene is responsible for the production of dyads observed in this series of mutant.

The Atps1-3 mutant exhibited a weaker phenotype than the two other alleles, whereas expression analysis suggested that this allele is also null. As this allele was in a different genetic background (Ws-4) to the two others (Col-0), we tested if this difference could be influencing the strength of the phenotype by introducing the Col-0 mutation into the Ws-4 background and vice versa. As expected for a background effect, the frequency of dyads increased with successive backcrosses when Atps1-3 was introduced into Col-0 (from 8% to 58% after four backcrosses) and decreased when Atps1-1 was introduced into the Ws-4 background (from 64% to 13% after four backcrosses). These results clearly indicate that the formation of diploid gametes is influenced by multiple genes, with AtPS1 acting as a major gene.

### Example 3

### Atps1 Mutants Produce Viable Diploid Pollen Grains

Pollen grain viability was examined by Alexander staining (ALEXANDER, Stain Technol, 44, 117-22, 1969) and showed that in the majority of cases the dyads and triads produced by the mutants result in viable pollen grains (more than 95% in the different Atps1 mutants: Col: 0 dead pollen grains out of 181; Atps1-1: 44 dead pollen grains out of 948; Atps1-2: 3 dead pollen grains out of 363). We did observe however that the pollen grains in mutant plants varied in size (data not shown). We then assessed the ploidy level of Atps1-1 and Atps1-2 pollen grains by quantifying spermatic nuclei DNA. Both mutants exhibited two different populations of pollen grains, one corresponding to viable haploid pollen grains (˜40% estimated by maximum likelihood) and another to viable diploid pollen grains (˜60% estimated) (data not shown). These proportions are compatible with the proportion of dyads, triads and tetrads observed in the mutants. In summary, the Atps1-1 and Atps1-2 mutants produce a high frequency of viable diploid pollen grains.

### Example 4

### Spontaneous Triploids Appears Among the Offspring of Diploid atps1 Mutants

As Atps1 mutants produce viable diploid pollen grains we search in the offspring of the diploid homozygous mutants the presence of polyploid plants by flow cytometry. Diploid and triploid plants (30%), but no tetraploid plants, were found among the progenies of Atps1-1 and Atps1-2 mutants (Atps1-1: 38 triploids out of 130 plants; Atps1-2: 30 triploids out of 103 plants). Flow cytometry results were confirmed by caryotyping a subset of 29 plants which were all confirmed to be triploid. This demonstrated that the diploid gametes produced in the Atps1 mutants are involved in fertilisation and produce viable triploid plants. The appearance of triploid, but not tetraploids, suggests that the Atps1 mutations only affect male meiosis.

To confirm that diploid gametes are produced only by the male side in Atps1 mutants, we realized reciprocal crosses between Atps1-2 mutants and wild type plants. As expected for the absence of a female meiotic defect we never isolated triploid plants when mutant ovules were fertilised with wild type pollen grains (0 triploids out of 182 plants). When mutant pollen was used for the cross we again observed that 30% of the progeny were triploids (20 triploids out of 56 plants).

The observed frequency of triploid plants (30%) among Atps1-1 and Atps1-2 mutant progeny is lower than expected from the frequency of diploid pollen grains produced by these mutants (˜60%). In parallel, more than 50% of the seeds obtained by selfing the Atps1-1 and Atps1-2 mutants, or backcrossing them as male, were abnormally coloured and shaped and germinated at a rate of ˜55%. Thus a potential explanation for the discrepancy between the frequency of diploid pollen grains and the frequency of triploid in the progeny, is abnormal seed development, which is commonly observed during crosses between plant species with different ploidy levels. These problems are related to the paternal to maternal ratio, which is very important for normal albumen development (SCOTT et al., Development, 125, 3329-41, 1998). The normal maternal to paternal ratio of albumen is 2:1 but in our case when a diploid sperm nucleus fertilised the two polar nuclei an aberrant 2:2 ratio was obtained. Nevertheless, it appears that approximately 25% of the triploid embryos were able to overcome this constraint.

### Example 5

### Atps1 Mutants are Affected in the Meiosis II Spindles Orientation

To unravel the mechanisms responsible of the production of dyads in atps1-1, we investigated the behavior of meiotic chromosomes in atps1-1 mutant and in the wild type (FIG. 4). Chromosome spreads showed that the meiosis in the Atps1-1 mutant progresses normally and is indistinguishable from the wild type until the end of the telophase I. Synapsis was complete, chiasmata formed (the cytological manifestation of crossovers) and bivalents were seen (compare FIG. 4G-I with FIG. 4 A-C, for example). At metaphase II, however, differences were seen compared to wild type with the 10 chromosomes aligned in a same plane, causing abnormal looking Figures, rather than two well separated metaphase II plates containing five chromosomes each (Compare FIG. 4J-K with FIG. 4D). In rare cases, metaphase II in Atps1 did appear normal however (FIG. 4L). At telophase II, we observed dyads (two sets of 10 chromosomes, FIG. 4M), triads (2 sets of five chromosomes and one set of 10, FIG. 4N) and normal tetrads (4 sets of 5 chromosomes, FIG. 4O). These observations are consistent with the previous finding that Atps1 meiotic products are a mixture of dyads, triads and tetrads.

These results and specifically the alignment of the 10 chromosomes at metaphase II suggested that the meiotic spindles in Atps1 mutants are defective at this stage. We thus examined spindle organisation by immunolocalisation with an alpha-tubulin antibody (FIG. 5). In wild type plants the majority of metaphase II spindles were roughly perpendicular to each other (FIG. 5A), leading to four well separated poles at anaphase II (FIG. 5B) and the formation of tetrads (FIG. 5C). In the Atps1 mutant, while individual metaphase II/anaphase II spindles appeared regular in most cases their respective orientation was aberrant. The majority of cells had parallel spindles (FIG. 5D to 50), fused spindles (FIGS. 5H and 5I) or tripolar spindles (FIGS. 5J and 5K). This defect in spindle orientation explains the appearance of triads and dyads. These conformations cause chromatids, that had been separated at meiosis I, to gather at anaphase II. Occasionally, three to four sets of chromosomes encompassed by a spindle were dispersed in the cell at metaphase H (FIG. 5L). This type of defect is probably the cause of the few unbalanced meiotic products observed in the Atps1 mutants.

The name AtPS1 for Arabidopsis thaliana parallel spindles 1 was chosen due to the high percentage of parallel spindles produced by the corresponding mutants.

### Example 6

### Dyads Production in Atps1 Mutant Results from the Formation of Parallel Spindles at Metaphase II

The apparition of parallel spindles at metaphase II in the Atps1 mutants appear to be leading to the formation of dyads. This proposed mechanism implies that unbalanced chromosome segregation at meiosis I would have no impact on the final distribution of chromosomes in the resulting dyad. To test this hypothesis we constructed a double Atspo11-1/Atps1 mutant.

The Atspo11-1 mutant (N646172 (Atspo11-1-3: STACEY et al., Plant Journal, 48, 206-16, 2006) obtained from the European Arabidopsis stock centre (SCHOLL et al., Plant Physiol, 124, 1477-80, 2000) displays an absence of bivalents at meiosis (MERCIER et al., Biochimie, 83, 1023-28, 2001) (FIG. 6A) leading to frequent unbalanced first divisions (FIG. 6B) that can be associated with lagging chromosomes (FIG. 6C). At metaphase II, unbalanced plates are seen (FIG. 6D), leading to unbalanced tetrads (FIG. 6E). Lagging chromosomes at anaphase II, lead to multiple metaphase II plates and then polyads with more than four nuclei (FIG. 6F).

In the Atspo11-11Atps1 background the first division was identical to the single Atspo11-1 phenotype. We observed 10 univalents at metaphase I (FIG. 6G), leading to missegregation at anaphase I, with two sets of unbalanced chromosomes (FIG. 6H) or three sets because of lagging chromosomes (FIG. 6I). At metaphase II, we regularly observed two unbalanced metaphase plates, which had a tendency to be parallel instead of perpendicular (FIG. 6J). This led to the formation of dyads which were always balanced (FIG. 6K to 6L, n=44). We also observed triads with one set of 10 chromosomes caused by an unbalanced first division followed by the fusion of two of the four second division products (FIG. 6N), which is highly consistent with our proposed mechanism. We also observed unbalanced tetrads (FIG. 6P), expected since the Atps1 mutation is not fully penetrant, and polyads due to lagging chromosomes at the first division (FIG. 6R).

Another prediction of the proposed mechanism is that centromere distribution should resemble that seen during mitosis, e.g. any heterozygosity at the centromeres should be retained in the diploid gametes. Indeed, in Atps1, the first division is identical to wild type, with the co-segregation of sister chromatids and separation of homologous chromatids. Thus, in the case of a heterozygous genotype, A/a, at the centromere, following the first division the two A alleles will end up at one pole, and the two a alleles at the opposite pole. In wild type, the second division separates the two sisters leading to four spores with one chromatid. In Atps1, the second division would regroup the products of the first division, thus grouping the a and A allele in each cell, leading to systematic heterozygosis at the centromere. Because of recombination, loci unlinked to centromeres should segregate randomly. We tested this prediction by taking advantage of the two genetic backgrounds of the Atps1-1(Col-0) and Atps1-3 mutants (Ws-4). F1 plants bearing the two mutations—thus mutant for AtPS1 and heterozygous for any Col-0/Ws-4 polymorphisms—were crossed as male to a third genetic background landsberg erecta (Ler). Caryotyping and genotyping of the obtained plants for trimorphic molecular markers (see additional methods) provided direct information regarding the genetic make up of the pollen grain produced by the mutant (FIG. 7). All the diploid pollen grains tested had the predicted genetic characteristics. They were systematically heterozygous at centromeres and segregating—because of recombination—at other loci. These results confirm that the “parallel spindle” defect is indeed the cause of at least the vast majority of 2n pollen in Atps1.

### Conclusion:

The above results show that mutants in the AtPS1 gene produce pollen grains which are up to 65% diploid and give rise to numerous triploid plants in the next generation. It is also shown Atps1 mutations only affect male meiosis and result in abnormal orientation of spindles at meiosis II. Since defects in meiosis II spindles are the main source of the 2n pollen which is extensively used in many plant breeding programs (for example in potato cf. CARPUTO et al., Genetics, 163, 287-94, 2003), the identification of a gene involved in these defects has important applications in plant breeding.

### Example 7

### Extinction of ATPS1 by RNAI Phenocopies the ATPS1 Mutants

An RNA interference (RNAi) hairpin construct was made based on a 361-bp cDNA fragment of the AtPS1 gene, represented as SEQ ID NO: 38.

The Prfb18 binary vector is derived from the pfgc5941 binary vector (GenBank: AY310901.1) by addition of GATEWAY® cloning sites on both sides of the chalcone synthase intron under control of the 35S promoter. The cDNA fragment SEQ ID NO: 38 and its reverse complement were placed in the GATEWAY® cloning sites of the Prb18 binary vector. The resulting vector comprises, downstream the 35S promoter, the reverse complement of SEQ ID NO: 38, followed by the chalcone synthase intron, followed by the cDNA fragment SEQ ID NO: 38, and by the octopine synthase polyA signal. This vector was used to transform Arabidopsis Col-0 wild type plants. The meiotic products of 14 primary transformants were observed. The results are summarized in Table I below.

TABLE I

| | % of | % of | |
|---|---|---|---|
| Genotype | dyads | triads | % tetrads |
| Col-0 | 0 | 0 | 100 |
| atps1-1 | 64 | 24 | 11 |
| atps1-2 | 65 | 20 | 13 |
| RNAi 35S AtPS1 #1 | 17 | 32 | 49 |
| RNAi 35S AtPS1 #2 | 4 | 8 | 84 |
| RNAi 35S AtPS1 #3 | 17 | 27 | 52 |
| RNAi 35S AtPS1 #4 | 20 | 16 | 60 |
| RNAi 35S AtPS1 #5 | 15 | 21 | 60 |
| RNAi 35S AtPS1 #6 | 62 | 16 | 20 |
| RNAi 35S AtPS1 #7 | 0 | 0 | 100 |
| RNAi 35S AtPS1 #8 | 0 | 0 | 100 |
| RNAi 35S AtPS1 #9 | 0 | 0 | 100 |
| RNAi 35S AtPS1 #10 | 0 | 0 | 100 |
| RNAi 35S AtPS1 #11 | 0 | 0 | 100 |
| RNAi 35S AtPS1 #12 | 0 | 0 | 100 |
| RNAi 35S AtPS1 #13 | 0 | 0 | 100 |
| RNAi 35S AtPS1 #14 | 0 | 0 | 100 |

These results show that while wild type produces only tetrads of spores, atps1 mutant and RNAi 35SAtPS1 lines produce a high proportion of dyads.

Among the 14 RNAi 35SAtPS1 lines, six produced a high frequency of dyads and triads instead of tetrads, a phenotype identical to the Atps1 mutant. FIG. 8 shows examples of meiotic products of a wild type plant, an atps1-1 mutant and a plant of RNAI35AtPS1#6 line.

The level of expression of AtPS1 in the Col-0 wild type plants was compared with the level of expression in three RNAi 35SAtPS1 lines (RNAi 35SAtPS1 #1, RNAi 35S AtPS1 #2, and RNAi 358 AtPS1 #6), producing different proportions of dyads. RT-PCR were performed on flower buds with primers specific of AtPS1:

| | |
|---|---|
| 5′ CCATAGTGAGAGTTATGGAGC 3′, | (SEQ ID NO: 39) |

and

| | |
|---|---|
| 5′ GGCGCCTTTTCAACCTCTACTTG 3′ | (SEQ ID NO: 40) |

or as a control, with primers specific of the ubiquitous gene APT.

The results are shown on FIG. 9. These results show that AtPS1 expression is reduced in the three RNAi 35SAtPS1 lines compared to the wild type (col).

Further there is a correlation between the efficiency of the AtPS1 expression reduction, and the proportion of dyads (cf. Table I) produced by the RNAi 35SAtPS1 lines.

Next, the ploidy level of the offspring of two RNAi 35S AtPS1 lines by flow cytometry was measured (results not shown). Triploids were found among the offspring of RNAi 35S AtPS1#1 and RNAi 35S AtPS1#6. This demonstrated that, like Atps1 mutants, RNAi 35S AtPS1 lines can produce functional male diploid gametes.

1. A method for obtaining a plant producing 2n pollen, wherein said method comprises the inhibition in said plant of a protein hereinafter designated as PS1 protein, wherein said protein:
comprises within its N-terminal region a domain having at least 40%, 45%, 50%, 60%, 65%, 70%, 75%, 80%, 85%, 90%, 95% or 98% sequence identity, or at least 60%, 65%, 70%, 75%, 80%, 85%, 90%, 95% or 98% sequence similarity with the FHA domain of the AtPS1 protein (amino acids 64-132 of SEQ ID NO: 1); and
comprises within its C-terminal region a domain having at least 40%, 45%, 50%, 60%, 65%, 70%, 75%, 80%, 85%, 90%, 95% or 98% sequence identity, or at least 60%, 65%, 70%, 75%, 80%, 85%, 90%, 95% or 98% sequence similarity with the PINc domain of the AtPS1 protein (amino acids 1237-1389 of SEQ ID NO: 1).

2. A method according to claim 1, wherein inhibition of said PS1 protein is obtained by mutagenesis of the PS1 gene or of its promoter, and the mutants having partially or totally lost the PS 1 protein activity are selected.

3. A method according to claim 1, wherein the inhibition of said PS1 protein is obtained by expressing in said plant of a silencing RNA targeting the gene encoding said protein.

4. An expression cassette comprising:
a promoter functional in a plant cell; and
a DNA construct selected from:
a) a DNA construct of 200 to 1000 bp comprising a fragment of a cDNA encoding a PS1 protein or of its complement or having at least 95%, 96%, 97%, 98%, or 99% sequence identity with said sequence encoding a PS1 protein;
b) a DNA construct which, when transcribed, forms a hairpin RNA targeting a PS1 gene; or
c) a DNA construct which, when transcribed, forms an amiRNA targeting a PS1 gene;
said DNA construct being placed under transcriptional control of said promoter.

5. A recombinant vector comprising an expression cassette of claim 4.

6. A transgenic plant producing 2n pollen, wherein said plant contains a transgene comprising an expression cassette of claim 4.

7. A method for producing 2n pollen, wherein said method comprises cultivating a plant obtained by the method of claim 1.

8. The method of claim 1 wherein the N-terminal region is selected from the 300 N-terminal amino acids, the 250 N-terminal amino acids or the 200 N-terminal amino acids.

9. The method of claim 1 wherein the C-terminal region is selected from the 350 C-terminal amino acids, the 300 C-terminal amino acids or the 250 C-terminal amino acids.

10. The method of claim 1 wherein said PS1 protein comprises within its N-terminal region a domain having at least 85% sequence identity, or at least 95% sequence similarity with the FHA domain of the AtPS1 protein (amino acids 64-132 of SEQ ID NO: 1); and within its C-terminal region a domain having at least 85% sequence identity, or at least 95% sequence similarity with the PINc domain of the AtPS1 protein (amino acids 1237-1389 of SEQ ID NO: 1).

11. The method of claim 1 wherein the PS 1 protein is a PS1 protein of a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.

12. The method of claim 1 wherein the N-terminal region of the PS1 protein comprises a FHA domain of a PS1 protein of a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.

13. The method of claim 1 wherein the C-terminal region of the PS1 protein comprises a PIN domain of a PS1 protein of a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.

14. The expression cassette of claim 4 wherein the PS1 protein or gene is a PS1 protein or gene of a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.

15. The expression cassette of claim 4 wherein the DNA construct is selected from a DNA construct which, when transcribed, forms a hairpin RNA targeting a plant PS1 gene; or a DNA construct which, when transcribed, forms an amiRNA targeting a plant PS1 gene.

16. The expression cassette of claim 15 wherein the PS 1 gene is a PS1 gene of a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.

17. The transgenic plant of claim 6 which is a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.

18. A method for producing 2n pollen, wherein said method comprises cultivating a plant obtained by the method of claim 2.

19. A method for producing 2n pollen, wherein said method comprises cultivating a plant obtained by the method of claim 3.

20. The method of claim 7 wherein the PS1 protein is a PS1 protein of a plant selected from Arabidopsis, Populus trichocarpa, Oryza sativa, Vitis vinifera, Glycine max, Sorghum bicolour, Solanum lycopersicum, Medicago truncatula, Solanum tuberosum, Helianthus argophyllus, Malus domestica or Triticum aestivum.


SEQUENCE LISTING (40 sequences)

SEQ ID NO: 1 - protein, 1477 aa, Arabidopsis thaliana
MEVKEEKLMEEKQRLPEKTIPVFTVLKNGAILKNIFVVNSRDFSSPERNGSTVSDDDGEVEEILVVGRHPDCDILLTHPSISRFHLEIRSISSRQKLFVTDLSSVHGTWVRDLRIEPHGCVEVEEGDTIRIGGSTRIYRLHWIPLSRAYDLDNPFVSPLDASTVLEQEEENRMLEAENLEVAQHQSLENTTSGDEGVLHLDVTSEGTGSSVPSEDEDTYVTTREMSMPVASPSVLTLVRDSVETQKLQFNEDLQTSPKWDLDVIESVAEKLSGSFVRSTQQSGGDVEGLGCSELFDAAEADECDVRGDGGLHLNVISEKMESSVPNMIEAENLEVAQHQSLANTALGDDEDLHLDVTSEGTGSSVPSEDEDTYITTMEISVPLASPNVLTLARDSIKTQKLQSTQDFQTPTMWDLDVVEAAAEKPSSSCVLGKKLSGGYVEELGCFELFVAAEADKCDVRGDGSLHLNEISERMESSMSNKEDDPFLAAKETSSLPLSTDFINPETLWLVEDVQASPEFCTSSVKANAENPSSGCSPSTEQIDGCFETSGCSAFDLAAEVESLSLHQEVSEETEFVTKEVMGVSSEPLGKADIRSHEENGESEDSRQVIEVSAEPVAKADIQSHEENGETEGSRQVIEVSPKSFSEAEPTIEILTGEAQGIIGSEFPSELAVETESENLLHQKSIGETKNEIRSHEDYGETEDYGETECSWPDIAVSPSSVSPPEPTLEILTDEARGLLGSEFLSEVTVETEIENLLHQKSNVETKADILIHEDYGETEVSRQIITVSPNSFSKAEPTLETEDSRQQARGLVGSDSEFQSEVAMKTECENLLNQKRNGETKVSSRQASPVSDCLSTPKDRLSSINTDDIQSLCSSSQPPSESEVNPATDQDQESGIISETEKPKTELLIGSGRSEKYYSLSEIEGEENTDIGRLSRCPIPSALAAKTSEDTKLIEELSSSDSGSQENQTPETHAVRDDVLCDMDSSSTCNIWSRRGKAASVLKIRTNKSQGKQKQTGRQPKDKLHRKQALSDKSISLTIHHGAEILEPEIFTPDKENLTPSSHMLKRLQDIGDVKDSKSSLKLSGKSCSSLVHSSIAVLASEAFTEPEIFTPDKENLTPSSHMLKRLREFGDIKDTKGSSSKATRKPFFDIRMEENVMVEQEPEDLHSLGSKSKLKHEPLAPKKKAERAPFQPLLEKSSFQSQSYTEASSTASARNNISRGIRSSSNLSDAKSKMKWTIVLDTSSLLDKESRKPLQLLQGLKGTHLVVPRTVLRELNEVKRSRSFLFRRRTEIASSALDWIEECKVNSKWWIQVQSPTEETKAIAPTPPVTPQSNGSSAFPFSLHWNNYAPEIDSPTSEDQVLECALLYRNRNRDEKLVLLSNDVTLKIKAMAEGVICETPHEFYESLVNPFSERFMWTESTARGRTWSHLDNDVLRERYNDRACRRKSTYNRGESGAAAKGLKLILLHNSHYGHTH

SEQ ID NO: 2 - protein, 1079 aa, Populus trichocarpa
MASNEEKKPEEEEEEERKIPVFTVLRNGAILKNIFVIDKSPLPSPTSSEPSIENEENPVQETEEILSFGRHPDCSIVLNHPSISRFHLQINSRPSSQKLFVTDLSSVHGTWVSGKKIEPGFRVELNEGDTIRVGGSTRYYRLHWVPLSRAYDMETPFISPLDMAMIEEKREENPVLEEENEAKMSQDENLVATERESVEEKGSLEVAGKDDERYQAMDSTSVENRETKSLDLILQDVGSLYCEEICESIAKKEILSAALVPDESMDSLFYDANEDIEISFRNDHNVKDILSPTTVQGVISETKCRQYDGHNQSPEYFSVRQELPETETKGSSMIRESNAVFSSLSTAEVESQSASEMLGATENGSLLRKGHEPINIFSHGIEMVNLSLPVKDLSENDSKKVRKENQTLEHLVALEPTYKEGNQGNFTANLLVNLNSACSDDQAVSLKSTYEEGNQEKFTANLLENLNSACSGDHAVALDPTYEEGTREHFTANLLENLNTSSCSDDHAVALDPTYEEGTQENFTTNLLENLNSSCSDDHAADDMLEVENQNLSRDDHGQSVYTSICSALLAAESVSSSFPVGLLSEIIDSKKCQTPESVLASIENQENLQSSHVRSEKKQSSRNIWSRRGKPKAVLQLQTSRSREKNRGDDVEWENQENIENRSISKTIFPGSEAAEEVLTPGKENYSPNTLLLKSLKKKGKREETQLSNSRRSTSSKIAFSPYKQPEEEMIASPDKENQTPKVLQQTKLAIPASRNQVKFKQEMVLEECKAERVPLQSLLVNFSGNSNSEASVPNDATRSSISVNCSQIMRKSNFTGDGKRRWTMVADTASLVDKESRKSLQLLQGLKGTHLVIPKMVIRELDCLKRRSSLFRKKTEASLVLEWIEECMVRTPWWIHVQSSMEEGRHIAPTPPASPQSRFSQGSEGFPCGTGSSVPFPAHGSFLEIVSPTAEDHILEYALSYRKMNRDGQLILLTNDVTLKIKAMSEGLICETAKECRDSLVNPFSERFLWADSSPRGQTWSVSDDLVLKERYYQSPSKKSSKGEGAKGLKLILLHNSQYGQISRSEQCSLFRNRYLF

SEQ ID NO: 3 - protein, 1112 aa, Oryza sativa
MASAAAEGEEAPIAAFAVSKGGVVLKNIFLNAPPSPLPVEEAARGRGGEEEDPPVMFGRHPECHVLVDHPSVSRFHLEVRSRRRQRRITVTDLSSVHGTWISGRRIPPNTPVELTAGDVLRLGSSRREYRLHWLSLPEAFDMEDLLPPLLEEDKEELSTCQEASKQLEPDQKESADTETHQETSQQVVSEQIDFHANVIPSAPPIPEFADLFALEESSVPEFDDSREGRIEGNLIEENHVIYSVESSITQPMLATVEDAGRSVKSGEKDTSNARRSKLKSVKTLRIETGRSKERITPLSYSYQKEENQNENPICSQNCGIECEACMVLFNNSYVGEAEEKEKMNILDRIMMEENQEQTNHLQSKEFVHYVAPLNLDYETFSDNENCVLSVAKETEHNDFNSVNCISQDSVCENPQKISELLHFVSPLVFKGDDFTDSKILQLCASVHKELSGPILENPFMQDISDENTNSNKDTGHEGLTLLNLDATLTSNENFAQSKIFVAPEDSESEGTISENLFEISNMKGNEENEENSPWDKENITPFVSGDIIVERSQLRLKPTTISQELMDSISPLNLEHNDFSDDESSILSIGEQMNSNELIAKNLIPLTSVDANMQKSHAGFMTIAHLDFKDSILTDEETSVLSPEKYDTISPVRQGNLFPDKENVTPASRDLKPIIGRKVLGPRVDNSLSVECTSKRRIHRQEPNELSAKSKVCHAVDDDVFYSDKENLTPISSGGIKARRCLPKSLTVDADQDQEAFYSDKENLTPVSSASRKTKDLSENRARMESTITKKRVVDRLPFQTLLSNSPLRHTSSLDSTQVNPRAVDVAMKLEGELNNVPHKGQESEKTKEGMKVWTMVTDMECLLDDESRKSIMLLRGLKGTQLVIPMIVIRELECLKKRERLFRMLSKATSMLQWINECMEKESWWIHVQSSTEMLPVAPTPPATPTALCNNGEREISAGTFNPIALFSPRSFSDIVSPKTEDRVLDCALLFNKLKGNQNIVILSNSVTLKIKAMAEGFPCEGAKEFRETLVNPCSSRFMWAASAPRGSAWSCLDETTLEENYYNSHHGARRRIPRPMEPAKGLKLILLHNSHYGQATNFVENRPLAPMASW

SEQ ID NO: 4 - protein, 869 aa, Vitis vinifera
MADENEKKIPVFTVLKNNAILKNIFVIDQPPPGISEPERPEHVLEEILMVGRHPDCNIMLTHPSISRFHLQIYSNPTLQKLSVMDLSSVHGTWVSEKKIQPRARVELKEGDIIRLGSSSRIYRLHWVPLSQAYDLENPFVSASDVLMEEEKEDEIYQDVSSFSVDSKEIQSQHPVLKGMESVFSDENCEPFVEKPIPSAPPEPENMNSSASDEEKTEGEGLPVVEAFEEIENQSPSRRDYEQTEILGAVNLLPSAEVLLETRNEQLDEEIKSPQPLFVSEVFSQGETPVGLPTKSWQKSKLLGSLDSYVADDKIEIPLVAEVLEEVENQSPPRKGYEQREASGLHSGAITTESVNSSVPDRNILSDIGNQQFSNENQPPKPLPVTLGLSDDENPESPPVRLEQKSSLPNIWSRRGKPASVLQIQTGRSTRKCIGDGNGAKIRKPKQEDLENKPISRALFPMLDGEETEIFTPNKENFSPNTLLLKSVNKKKGILEETKQSTLCRSSSSKFSTGPNKCSEEDTSTFSDKENQTPQVLQTRKSVRPSPENSSRNRGKLEKEIMVMKRGAERVPFHSLLENAACKSKSEVSILGAKTRSSNSVNCTGTTGNATNSSFNNSAGEGKRRWNMVVDATCLLNKESRKSLQLLQGLKGTQLIIPRMVIRELDCLKRRGSLFRRISEVSLVLQWIEECMVKTKWWIHVQSSIEEGRPIAPTPPASPPRFSEGSGGFISGTTSSVPFSACGSLMEIVSPTAEDHILECALFFRRIKNDGQLVLFTNDVTLKIKAMAEGLNCETVEEFRESLVNPFSERFMWSDSSPRGQTWSYLDDVVLREKYYRCPLKKASKGGESAKGLKLILLHNSHYGKIGSIS

SEQ ID NO: 5 - protein, 1123 aa, Glycine max
  misc_feature (700)..(700): Xaa can be any naturally occurring amino acid
MAEKKNPEQEEQHRFPVLTVLKNNAILKNIFIVLDEHDEDQTVLIGRHPNCNIVLTHPSVSRFHLRIRSNPSSRTLSLVDLASVQGTWVRGRKLEPGVSVELKEGDTFTVGISTRIYRLSWAPLTQLGVVVPQQHQKEDEQENIIKDENLEHTAEQDIPMSEDIVSVCCDEERKIHSEDEALGVPNGTETSCFPTNSCVENIICDCQLSPPYIQSPPCAQPVDELDNTKKIEARLEVEMPGETNLLCTLREYLKHNICLPVVEAVQGTKMQQFQAPHDTFTGQPPSLEMHWSSFQINIDPSSFDEKHAAAVPVIPTESEFGCTHGDIDKVEGILTTAPRSFNSENTCLIVDEDIPDSEFHQMEVVEEVSVDSVPDGEKQDEMHGSSSPTNLDPAFLDEKHVAAVAIIPTESEFGCTYGDNDKVEDILTTGSRTFNSENTCLIVDKDIPDSEFHQMEVVEEISVDSVPDEEKQDECDEENLNGKSCREEGYSLDEVVEDNGNKCIKNIDPASFDGKGLTAVTVIPTEFEFGCTLGDNERIEDILEMESRTINSENTSLLDEKAIAVTKFQLVNIVEEGGYSLDEVVEDNGNKCIKNIDPASFDGKGLAAVTVIPSESEFGCTLGDNERIEDILEMESRTINSENTSLLDENAIAVTKFQVVNIVEEVAMDSISDGNKCIKNIDPASFDGKGLAAVTVIPSXDKCGKELESKLPASLNAKSCHEQGKSVAEIAEDTGKKCASSISSTSFQVESPNSSMPREQTPQSLTAVTRCSGGEFLENHVKPTEKSSAFGSIWSRCKPASAPLVQARKSRFMSTAKVGTEVKRSNEKNVVINKLMPKDLSAVFDEEKEAFILNKENLSPNTYHLQFMRKKDKPEEIKHSISQRSPNLSYFSPRIYLDKRISSKRSWDMVVDTASLLNKESRKALQLLQGLKGTRLIIPSLVIRELGSMKQKFRIFRTTSEASLALEWIEECLEKTRWWIHIQSSMEEFRLTALTHHASPQTRFIEESWAFPGLNTLKKCASPKVEDHILDSALQYGRKENVGQLVLLSSDVSLKIKSMAKGLLCETVQQFRQSLVNPFSERFMWPKSSPRGLTWSCQDDLVLREKYCGLPSKAGLKLITFHD

SEQ ID NO: 6 - protein, 1198 aa, Glycine max
MGLENQSKAEEEKEGEIPVLTVLKNNTVLKNIFIVNKPTDQKQSSADHVNVLLVGRHPDCDLMLTHPSISRFHLQIRSNPSSRTFSLLDLSSVHGTWVSGRRIEPMVSVEMKEGETLRVGVSSRVYRLHWIPVSRAYDLENPFVAQLDSVAEEEEEEKEEEEEMQNLSCCPAEMEEIESMDSIVEDISSLFLDENVELTVKEEIPLEPWMLEDMISLCCEEERKSPSKEEAIEIPSDPFGTETSYLPTISDGENNLCDSVSQVLSPTYVESLVECDDTLTENLSDTSCLPAVEAVLETKMLQFHTPPDIFTSPLPSGHENLFEKHYSSLPVNTAPSSLGEKSAPEAVIMPEETECESEDDESIIDIFTVPESLHAAEDVITTNESESECTLRDDGSVTDAFIAGAGNFNSEDVFLPVEEVMPGTKVEQIKIVEKVAMDSLSDEGKQHMYRSLSQLLNDKFCHDQWHSLNEIVQDVRNKHAYSISPTPHQIESVNLSMPQEVVLNIMNEDQTQHSDMENLESCIKAMEKTSTNIWSRRGKATSAPQVRTSKSILKNAANVEVAMSNEKDIRNRTISKNLSSVLDGEVEEDDEEIYTPDKENISPNTLHLRFLKKGKIEGIKHSKSQRSRHILRDTFNCDIYPNESIDPTLCNMNKKDLFSVLDGEVKEKEIFIPEEENLNPNALQLRLLKKKGKVEEIKRSKSRRSPLSKGTFNPDMYPNENIGSTLCNINQKDSINRTISRDLFSDLEGEEEEEIFTPDKENFSPNTLHLRLLKKKGKVEEIKHSKSQRSPLSKGTFNPDMYPNESIGPSLRRMNQKDVINKTISKDLLSDLDGEEEEEEIFTPDKENFSPNTLRLQLLKKKDNFCPNLYPDENIIPTSNEENQTLKGVQDQKLQRNPFSSHIKFAQEQDLKDRVERIPFQSLRNSGDKRRSGTCCPVSASKSLHFSNCGQILDQRFNPSDISGVPKKRSWDMIVDTTSLVNKESRKALQLLQGLKGTRLIIPRLVIRELDRMKQQFTIFRRISESSLALEWIEECMVKSNWWIHIQSSVDEGRLIAPTPPASPLTQFSEESWTSLSTQKFSMEIASPTVEDHILDFALLYRRNQNDGQLILLSEDVTLKIKCMAEGLLCEPVQEFRESLVNPFSERFLWDKSIPRGQTWSCQDDVVLREKFCRLRKPSKGVASGLKLILLHNSQYGL

SEQ ID NO: 7 - protein, 1092 aa, Sorghum bicolor
MAAAAADGDAPIAAFAVAKGGVVLKHIFLNAPPPEAATTRGRGAEDSEDEEEDPPVMVGRHPDCHVLVDHPSVSRFHLELRCRRRQRLITVTDLCSVHGTWVSGRRIPPNTPVDLATGDTLRLGASKREYRLLWLSLREAFEMDDLMYMPSLPEEDKEEREPHAYKEPNSQLVPGHRDSVGMETHQDTSEQIVSEDITFPAKVAPSAPPLSDFVHPFLAEEPSLSQFHEKIDGVTEEKLVEKNQFSESFGSLIIQEMPGTLTNAGKSIHSDKKDASNKMSKRSKLRSVKSLCVDTGRSRDRSSTLIHSIRKGDQNEILVCSQSCGTECTACIALFGISEVERAEEKEELIAEDKVDMNPPASMIMEGTMKERKPENYIPQDPVDAKLQKKVGLLDSALPLHFKDDAFTDKEIPEWNGATIDTESVLVSENLIMPEMKHDGLNHLNLEGDLSENENMDPNNVAEGPGNCNLEGTICGNLFDNLDTEGIEEDEEICPMDKDEITPNVSGNIIMERSHIGLKPTISQQLMDSISPLNLDHDDFSENENSKLYTGDQMKSNEPVSENLNPLMPISHLEFKDDILLDMENSVPALEKSEAMAVRQENLFSEKENVTPASKVKTNVRRVLGTRMDNSMSAAADSNKKKVLGSRVDNSVSTENSSNKKQCSELSSKSEKFHTVDFDVFYSDKENLTPIASGGMKARKCFPNDLSVDLDQDQEAFCSDKENLTPLSSAARKTRDMSENRARVESAITKKRVADRLPFQTLLSNSPLRPASSHDCTCAVAGPTDITAGDLVIKLEDKFNNLSCNNQESGSAGQGMKTWTMVANTDSLLDDESRKAIMLLKGLKGTRLFIPRIVIRELDSMKQREGLFRRSTKATTILQWIEECMATESWWIHVQSSADMFPVAAPTPPATPSAQRIDEEMEISSSSSTFNPMASFFSPRSSPALADIVSPRPEDRVLDCAILVSRLRRSGEKVVVLSHSVSLKIKAMAEGLPCEGAKEFRESLMDPSSRRFMWAASAPRGAACSCLDASALAENYYNSHHHAMKRSGVVPAAAARPAQAAKGLKLILRHNSLYAQATTETPPLLASALASV

SEQ ID NO: 8 - protein, 1096 aa, Zea mays
  misc_feature (1033)..(1033): Xaa can be any naturally occurring amino acid
MAAADGDALIAAFAVSKGGIVLKHIFLNAPPPEAMCGSGGREVESDEEDPPVMVGRHPDCHVLVDHPSVSRFHLELRCRRRQSLITVTDLHSVHGTWVSGRRIPPNTPVDLATGDTLRLGASKREYKLLWLSLREAFEMDDLMYMPSLPEEDKEEPYVKEPSSKLLPGHRDSVNMETHQDTSEQIVSEDIAFPAKVAPSAPPLSEFLQPFFVEEHSLSQFHEKRNGVTEEKLVDKNQISESFGSLIIQEMPGTLTNAGKSIQSGEQEDASNKVSKRSKLKSVKSLRVDTGRSSERSSTLSHSFQKGDQNDIVVCSQSCGTECAVCIALFGISEIEKAEEKEELIAEDNVDMNPPASMIMEGNMNERKPDNYIPQDPIGAKLQKKLGLLDSALPLHFKDDVFADKEIPQWNVASVHTESELLSEYLIIPEVKHDDLNHLNLEEGLSKSENINPNKITEGPGNCQLEGTIRGNLFDNLDTDGIEEGEEICPLDKDEITPNGSGNIIMERSNIVLKPTISQQLMDSISPLNLDHGDFSENENSMLNTGDQMKLNEPVSENLNPLIPTDEKYLKSQTEECMPISYLEFKDDILLDRENSVLAPRKYEAMSPVRQENLFSDKENVTPASKVKTVVRGVLGTRMDNSVSAANASNKNKVLGSRVDNSVSTENSSNKKQCELSSKSKKVHTVDFDVFYSDKENLTPISSGGMKARKCFPKDLSVDLDQDQEAFCSDKENLTPLSSAARKTRDMSGNLTRVESAVTKKRVVGRLPFQTLVSNSPLRPASSHDCTCAVARPAGVAAGDLAIKLEDKLNDLSCNGHESGSAGEGMKTWTMVANTDSLLDDESRKAIMLLKGLKGTRLFIPRIVIRELDSMKQREGLFRRSTKATSILQWIEECMARESWWIHVQSSADMFPVAPTPPATPSAQRIDEEIEISSGSFNPMMALFGPRSSAALADMISPRPEDRVLDCALLVSRVRSNEKVVVLSNSVTLKIKAMAEGLPCEGAKEFRESLVDPSSRRFMWAASAPRGSAWSCLXASALAENYYNSRHHAMKRRVLVAARPSESEAAKGLKLILRHNSLYAQATDAVNKTPLVSLAAV

SEQ ID NO: 9 - protein, 1226 aa, Medicago truncatula
MASIVVSDDNPNQQQQSLIPVLTVFKNNSILKNIIILNNNNNNNKYNDDQILLVGRHPNCNIVLFHPSISRFHLQIRFNPSSRSISLLDLSSGISEKPLFSFFFFEYSFFFICYSFLESWFFIVCGTVHGTWVCGRKLEHGVSVDLKEGDTFQLGSSSRVYLLQFVMLSLVFVFLSFGFGLQNIGSLGCDDKRKDQSNDETFEDENDSFGTETSCCNGENKLCGCHFCLLSPPYTQSVDETDNIQMGEACPEVEMPGETNLFCTLRECFQQNICIPVAEAVQGSKLHQQSSAEKQLIDPESSFGEKGDGAVDEVPKESEFEGTFEYIVTTGGRVFNSEDMPCSESHQTNTNEEVSVDSLSDGEKQGSCGEEYESELQNLNANSCHKQQYSPDEIVEDIGKQCIENMDPASSEENGVAALSVTPKEHKLEFFSEENDMIDDVLSSVARFFNSENTSSLVKETIHHVTNFQQINTVEEVAAVDSLSDEEKENKCDVEFKAYLNIKPCDEEGNSLVETVEETVKSFQTESVNPLSVNTYSLVEDSIPVTNFQLINIVDEVATVDSLSDEEKENECDEEFKAYLYVKPCDEGSSLDETVEETVKSFQTESLNPSSGNTSSLVEEAIPVTNFQLINIVEEVATVDSLSNEEKENECDEEFKAYLYVKPCDEEGNSLDETVEETVKSFQTESLNPSVTQETDLEITEKKENQTLQSLVAVAGCFDVKFHENCVEESVEGSLTLGSDILSRRDKAASAPQDRTRKSRLLNTPDVDTKFVMSNLKDINIINKPMPQNIFSDLDEEEMFTPNKENSSPTNTFHSQFMRKKGVLEESKSSKSQRAHNLKASFSSIIYSAERCTSAISNKENQTPKSQRAHNLKASFSPIIYSAERSTSAISNKENLTPREAREWKSQRSHNLRASFSPIIYSAERSASAISNKENLTPKEALEWMSGRNPLECRNTMELRKKRVERMPLQSLISSGGNHNSNSSPFSSSPFSAAKSILGVTVRSSNCGHISDKHAQPSVRYHKAERKRSWDLVVDTSSLLNKESRKALQLLQGLKRTRLIIPQSSVIRELGSMKQQIGIFRRISEAALALEWIEECIGKTKWWIHIQSSMEDEFRLIAPTPPTQFNEDVLDCALQYRRKDNVGQIVLLSDDVNLKIKSMAKKKLLQGLLSETVQQFRQSLVNPFSERFMWANSSPRGLTWSCRDDVVLREKYCCLPSKAGLKLLAT

SEQ ID NO: 10 - protein, 878 aa, Solanum lycopersicum
MADKLEISSIEEDKKIPVFTVLKNGAILKNIFLLDNPPPCSNQESEIEEILVVGRHPDCNITLEHPSISRFHLRIHSKPSSLSLSVTDLSSESSYMVLDHHLMIWGSKSSDLLICSGKVPKPTCGISLVHGTWISGKKLESGVKVELKEGDRMQLGGSSRVYRLHWVPISHAYDLENPFVPTLCESEPEESTQEEQHQDESGFSLQNDQIQKEDYDMVQGLDSSFSGMSSLPRLRSLTPPAPPMLDKNVAANENLPGNIHEEGEISLRQPAYQADKENSIPEALLVPGQSPNENADGTPPRSQQRCSSIWSRRGKHSNVQIQTGKDRAMNENIDMETEVESINREIEGTISVSKDLFASGNKDKEEEVFTPDKENHTPSSLFLGSMKKSCLSEMTNRSGRKSVLSNMDETDEETFTPDKENMTPETRRLRLMKKIGSQHQIKHPKLFKSSSLKLVVEPRSNQAAGCVSHKKEKLGSTTKSTQPNVDENDEEIFTPDKENMTPDTRLMRSMKKIGKLEDLKLESFKFSLDNVVDPIFHQNGTPFSSEKDNLNDKVLEEQKSTILAPRYPARLEVNTVKNRMDRVPLQSLLVNYPVKTSSISPEENIKLRDYPIQHPETMELCPFFNESFMEKKRWTIVVDTGSLLNKESRKSLQLLQGLRRTYMIIPRTVIRELDCMKRRASLFRRTTEVSAALEWIEDCMINAKAWIHVQSCAEETRAVAPTPPATAPLSLFSEENGMFPVGSHQFSPHSGLMDFASPTAEDHILEYALFFKRTNRNGQLVLLSNDLTMKIKAMAEGLNCETAEEFRESLVNPFSERFLWKDSSPRGRTWSCEDDFVLRETYYHGPPKKPSMSGEAAKGLKLILLHNSHFRCHISTAS

SEQ ID NO: 11 - protein, 153 aa, Solanum tuberosum
FEEILVVGLHPDCYIVLEHPSISRFHLTIHSNPSSHSLSVIDLSSVHGSWISGNKIEPGVRVELKEGDKMKLGGSRREYMLHWIPISRAYDLENPFVAPLCEEEPFKEMDEKEHHDENGFALHNEGDDLVQDQDSSFSCPSLLPYIKCPTPSA

SEQ ID NO: 12 - protein, 132 aa, Helianthus argophyllus
PQSLKYLKLLEDIEGTQLFLTKTVVRELMDIESQDNFFNRSSKKASLALEWIDECMMNTSQWIHMDDDDDETVRRSSTVLEIALRLREEDSDQKIIILSDNLTLKIKALAEGIMCEAAEEFHKSLVNPFSER

SEQ ID NO: 13 - protein, 189 aa, Malus domestica
IRRSPMHALINLVGEGKMSWTMVADATTLLDKESRKSLQFLQGLKGTQLIIPRMVIRELDCLKQRGSLFIKKTEAELVLEWIKDCMIKTNWWIHVQSSMEDGRLIAPTPPASPQSLFNEKSWGFPSRTTGSLTFSRCGSMMDLVSPSPEDHILDCALLCRRMKRNYGQPVLLSTDVALKIKAMEEGLLC

SEQ ID NO: 14 - protein, 200 aa, Triticum_aestivum
KVWTMLADTDCLLDDESRKSIMLLKGIKGTHLIIPRIVMRELEGMKQREGMFKRSSKATSIMQWIEDCMENESWWIHVQSSSEMLPVAPTPPATPTETQRNSEESEATAAGAFNSMLALFSPRSFTGIFSPRILADIDSPKTEDRVLDCALLFNKLRGCGQNMVILSNSVNLKIKAMSEGLLCEGAKEFRETLMNPCSER

SEQ ID NO: 15 - DNA, 19 bp, Artificial
  PCR primer
acatctcccttgtcgtaac

SEQ ID NO: 16 - DNA, 19 bp, Artificial
  PCR primer
atctctcaatcgttcgttc

SEQ ID NO: 17 - DNA, 25 bp, Artificial
  PCR primer
ctgataccagacgttgcccgcataa

SEQ ID NO: 18 - DNA, 20 bp, Artificial
  PCR primer
tcggagtcacgaagactatg

SEQ ID NO: 19 - DNA, 21 bp, Artificial
  PCR primer
cagtctcactgattattcctg

SEQ ID NO: 20 - DNA, 21 bp, Artificial
  PCR primer
gctttcttcccttcctttctc

SEQ ID NO: 21 - DNA, 21 bp, Artificial
  PCR primer
aaggctgatattctgattcat

SEQ ID NO: 22 - DNA, 21 bp, Artificial
  PCR primer
ctcttgttggtccgtatctta

SEQ ID NO: 23 - DNA, 28 bp, Artificial
  PCR primer
aacgtccgcaatgtgttattaagttgtc

SEQ ID NO: 24 - DNA, 21 bp, Artificial
  PCR primer
aatcggtgagtcaggtttcag

SEQ ID NO: 25 - DNA, 21 bp, Artificial
  PCR primer
ccatggatgaaagcgatttag

SEQ ID NO: 26 - DNA, 19 bp, Artificial
  PCR primer
tcctttcatcttaatatgc

SEQ ID NO: 27 - DNA, 19 bp, Artificial
  PCR primer
tctgtccacgaattattta

SEQ ID NO: 28 - DNA, 16 bp, Artificial
  PCR primer
cccatgtctccgatga

SEQ ID NO: 29 - DNA, 19 bp, Artificial
  PCR primer
ggcgtttaatttgcattct

SEQ ID NO: 30 - DNA, 20 bp, Artificial
  PCR primer
gttttgggaagttttgctgg

SEQ ID NO: 31 - DNA, 24 bp, Artificial
  PCR primer
cagtctaaaagcgagagtatgatg

SEQ ID NO: 32 - DNA, 21 bp, Artificial
  PCR primer
gaggatacctcttgctgattc

SEQ ID NO: 33 - DNA, 21 bp, Artificial
  PCR primer
cctggccttaggaacttactc

SEQ ID NO: 34 - DNA, 22 bp, Artificial
  PCR primer
caatttgttaccagttttgcag

SEQ ID NO: 35 - DNA, 24 bp, Artificial
  PCR primer
tgagtttggttttttgttattagc

SEQ ID NO: 36 - DNA, 20 bp, Artificial
  PCR primer
gccttttcaacctctacttg

SEQ ID NO: 37 - DNA, 23 bp, Artificial
  PCR primer
atggtgatagatgatgatgatac

SEQ ID NO: 38 - DNA, 362 bp, Arabidopsis thaliana
gggaactaaatgaggtgaagcgcagtcgcagttttctctttagaagaagaacagagattgcttcttcagctctggactggatcgaagaatgtaaggttaattcaaaatggtggattcaagtccagagtccaacagaagaaaccaaagcaattgcaccaaccccaccagtcactccccagtcaaatggctcatcggcattccccttttcacttcactggaacaactatgcaccagagattgattctccgacatcagaagatcaagttctcgaatgtgctcttctttatcgaaaccgtaaccgtgacgaaaaactcgttcttcttagcaacgatgtaactctcaagatcaaagccatggcagag

SEQ ID NO: 39 - DNA, 21 bp, Artificial Sequence
  PCR primer
ccatagtgagagttatggagc

SEQ ID NO: 40 - DNA, 23 bp, Artificial Sequence
  PCR primer
ggcgccttttcaacctctacttg